(19) United States
(12) Patent Application Publication
(10) Pub. No.: US 2014/0371771 A1
(43) Pub. Date: Dec. 18, 2014

(54) SYSTEM AND METHOD FOR RESECTING CORNEAL TISSUE

(71) Applicant: AMO Development, LLC., Santa Ana, CA (US)

(72) Inventors: Ronald M. Kurtz, Irvine, CA (US); Gagik Jotyan, Dorog (HU); Tibor Juhasz, Ladera Ranch, CA (US); Melvin A. Sarayba, Ladera Ranch, CA (US)

(21) Appl. No.: 14/470,721

(22) Filed: Aug. 27, 2014

Related U.S. Application Data
(63) Continuation-in-part of application No. 11/469,941, filed on Sep. 5, 2006.

(51) Int. Cl. A61F 9/008; A61F 9/009; A61F 9/013

(52) U.S. Cl. CPC A61F 9/00831; A61F 9/013; A61F 9/009
USPC 606/166

(57) ABSTRACT

A system and method for resecting and transplanting corneal tissue is disclosed. In a recipient cornea, a resection depth from the anterior surface of the recipient cornea is determined based upon a biomechanical model of the recipient cornea. A resection incision for resecting a posterior portion of the recipient cornea is made at the resection depth. Preferably, the incision is made using a surgical laser. Optionally, a contact lens may be placed against the anterior surface of the recipient cornea, wherein the shape of the anterior surface is conformed to the shape of the contact lens.

### BACKGROUND OF THE INVENTION

1. Field of the Invention

The field of the present invention is systems and procedures for transplanting corneas.

2. Background

Many different diseases or conditions of the cornea exist which completely or effectively rob those who suffer from such diseases or conditions of vision. Fortunately, corneal transplant procedures, which are becoming more commonplace, are capable of substantially restoring lost vision. Currently, there are many techniques and tools for performing corneal transplantation. Unfortunately, all suffer from inherent limitations or risk of complications.

A procedure called posterior lamellar keratoplasty (PLK), or deep lamellar keratoplasty, was previously developed to address these shortcomings for patients with endothelial cell dysfunction. In this procedure, a thin posterior or lenticule of stromal tissue (along with descemets membrane and endothelial cells attached) is removed from the cornea of a diseased eye. A similar procedure is performed on a donor eye to obtain donor tissue. When the donor tissue is placed in the recipient's eye, no sutures are required because the anterior surface of the recipients' cornea was left intact. One advantage of this procedure is that it results in little post-operative astigmatism. While PLK has its advantages, in practice it is difficult to perform, regardless of whether manual or automated instruments are used.

A procedure called descemets strip endokeratoplasty was introduced in attempts have been made to improve on PLK. In this procedure, the stromal tissue is not removed from the recipient's cornea, rather only the descemets membrane and endothelial cells are removed. While this simplifies the procedure, it results in an abnormally thick post-operative cornea and may degrade optical quality. In addition, there is the added risk of the additional tissue becoming dislodged from the recipient's cornea since no cavity was created into which it can be fully inserted.

Other attempts have been bade to improve on PLK using femtosecond lasers to incise both the recipient's and donor's cornea with high precision. While this makes PLK more practical, it also introduces some new difficulties into the procedure. Specifically, contact lenses, both flat and curved, are generally used with femtosecond lasers in order to provide proper registration of the cornea with the focal point of the pulsed laser beam. These contact lenses distort the shape of the cornea by forcing the anterior surface of the cornea to conform to the curvature of the lens. This distortion of the cornea introduces folds or wrinkles into the corneal tissue, with the folds being more pronounced in the posterior portion of the cornea. Thus, when a resection incision is made, the folds or wrinkles can cause the incision to have an irregular surface when the cornea returns to its natural curvature if the incision is made too near the posterior surface of the cornea. Such an irregular surface, however, can have adverse effects on wound healing and optical quality of the post-operative cornea.

Another problem arises when the resection incision is made too near the anterior surface of the cornea. Under such circumstances, the surgically repaired cornea may begin to weaken and deteriorate due to ectasia because the anterior portion was overly thinned during the procedure.

### SUMMARY OF THE INVENTION

The present invention is directed toward a system and method for resecting corneal tissue. In the system, a surgical laser emits a pulsed laser beam which is directed into the cornea by a focusing assembly. An interface is programmed with a biomechanical model of corneal tissue, which is used to provide a depth range for selection of a resection depth from an anterior corneal surface. The selected resection depth is received by a controller which employs the focusing assembly to move the focal point of the pulsed laser beam and make a resection incision at the resection depth for resecting a portion of the cornea.

In the method, the resection depth is first determined using a biomechanical model of the cornea. Thereafter, a resection incision is made at the resection depth for purposes of resecting a posterior portion of the cornea.

Other options may be added to the above system or process, either singly or in combination. In a first option, the resection depth is determined using the anterior surface of the cornea as a reference. In a second option, a contact lens is placed against the anterior surface of the cornea. The contact lens is adapted to conform the anterior surface of the cornea to the shape of the contact lens, which preferably has a radial or planar form. With the contact lens in place, the resection incision is made using a surgical laser. In a third option, the resection depth is selected to minimize surface irregularities resulting from the resection incision. In a fourth option, the resection depth is selected to minimize post-operative weakening of the cornea. In a fifth option, the resection incision is placed at a uniform distance from one of the posterior surface of the cornea or the anterior surface of the cornea. In a sixth option, a posterior portion of corneal tissue is resected from a donor cornea, and this donor tissue is grafted into the recipient cornea. In resecting the donor tissue, the ratio of the depth of the resection incision in the recipient cornea, as compared to the overall thickness of the recipient cornea, is first determined. From this ratio, the depth of the resection incision in the donor cornea may be determined. This is done by making the depth of the resection incision in the donor cornea have the same ratio as compared to the overall thickness of the donor cornea.

Accordingly, an improved system and method of resecting corneal tissue are disclosed. Advantages of the improvements will appear from the drawings and the description of the preferred embodiment.

### BRIEF DESCRIPTION OF THE DRAWINGS

In the drawings, wherein like reference numerals refer to similar components:

FIG. 1 is a flow chart illustrating steps for performing a corneal transplant procedure;

FIGS. 2A & 2B illustrate resection incisions in a recipient cornea and a donor cornea, respectively, as part of a corneal transplant procedure; and

FIG. 3 is a schematic view of a system for resecting corneal tissue.

### DETAILED DESCRIPTION OF THE PREFERRED EMBODIMENTS

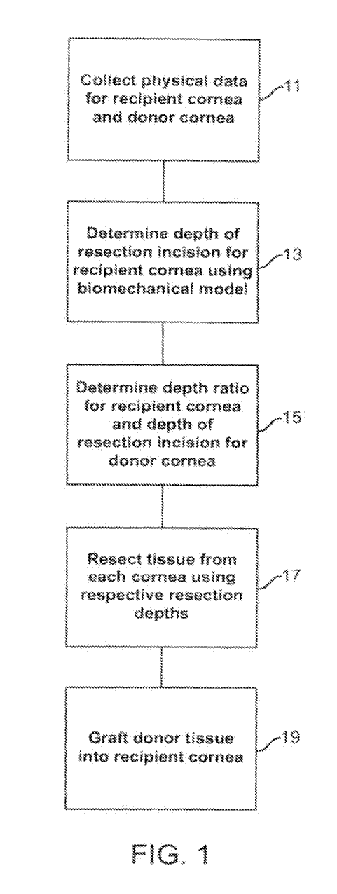

Turning in detail to the drawings, FIG. 1 is a flow chart illustrating a process for performing a PLK procedure. The basic steps of the PLK procedure are (1) collect physical data 11 for both the recipient cornea and the donor cornea; (2) determine the depth of the resection incision 13 for the recipient cornea; (3) determine the depth ratio of the resection incision 15 in the recipient cornea and the depth of the resection incision in the donor cornea; (4) resect tissue 17 from both the recipient and donor corneas; and (5) graft 19 the donor tissue into the recipient cornea. Each step is explained in further detail below.

The physical data collected includes thickness measurements of both the recipient and donor corneas. These thickness measurements are used to develop a thickness profile of each cornea. Additional physical data may also be collected for each cornea, with the type of data collected being dependent upon the requirements of biomechanical model used in the subsequent step of the PLK procedure. This thickness profile, along with any other data needed for the biomechanical model, may be obtained by any one of the many known methods for measuring the physical structure of the eye, with the preferred method being through optical coherence tomography (OCT). Many commercially available OCT scanners are capable of performing such measurements. One example is the Visante™ OCT scanning system, manufactured by Carl Zeiss Meditec, which has an office in Dublin, Calif. One advantage of the Visante™ OCT system is that it does not make contact with the cornea when performing the OCT scan.

Following collection of the physical data, the depth of the resection incision for the recipient cornea is determined using the biomechanical model. Preferably, the biomechanical model takes into account stresses introduced into the corneal tissue when the cornea is conformed to the shape of a contact lens set against the anterior surface when the resection incision is made with a surgical laser system. Such stresses can cause folds in the corneal tissue which are more pronounced in the posterior regions of the cornea. A resection incision made in areas with more pronounced folds can lead directly to surface irregularities at the resection incision when contact lens is removed and the cornea returns to its normal shape. Therefore, the biomechanical model is used to place the resection incision at a depth which minimizes or eliminates surface irregularities at the resection incision.

The biomechanical model also preferably takes into account the post-operative stability of the recipient cornea when determining the depth of the resection incision. Without sufficient post-operative stability, the cornea may undergo weakening through pathological deterioration, e.g., ectasia, thereby exposing the recipient to a risk of vision impairment. Maintaining sufficient tissue between the anterior surface of the recipient cornea and the resection incision is an important factor to maintaining post-operative stability. The biomechanical model is therefore employed to determine the minimum depth from the anterior surface at which the resection incision is likely to cause post-operative stability problems.

Optionally, the biomechanical model may be developed to employ a database of corneal measurements collected from many patients. Using information from the database, together with at least the known curvature of the contact lens, such a biomechanical model could be used to determine an appropriate depth for the resection incision.

The resection depth selected for the recipient cornea is selected to be between the minimum depth needed to create post-operative stability in the recipient cornea and an appropriate depth to avoid or eliminate surface irregularities at the resection incision. In the event that there is no region between the two extremes, maintaining post-operative stability in the cornea is preferred. In such instances, the radius of the contact lens upon which the shape of the cornea is conformed for purposes of the PLK procedure and the biomechanical model could be enlarged to reduce stresses on the corneal tissue and help create a region in which post-operative stability can be maintained along with minimizing irregularities at the resection incision.

The biomechanical model may be one of several previously developed models which are known to skilled artisans, or it may be one which is specifically developed for the PLK procedure. Modeling software, such as the software published by Structural Research & Analysis Corp. of Santa Monica, Calif. under the title “Cosmos/M”, may be used to develop biomechanical models. One such model is described in the article by G. Djotyan et al., “Finite Element Modeling of Posterior Lamellar Keratoplasty: Construction of Theoretical Nomograms for Induced Refractive Errors”, Ophthalmic Research, Vol.38, n.5, 2006.

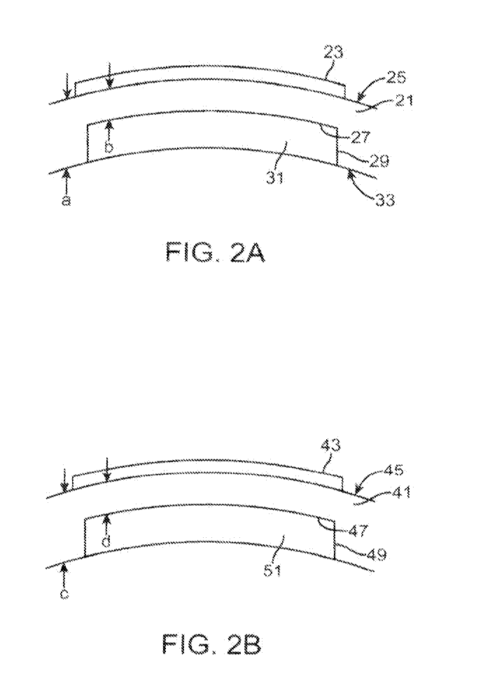

FIG. 2A illustrates the recipient cornea 21 with a contact lens 23 placed against the anterior surface 25 of the recipient cornea 21. The resection incision 27 and the sidecut 29 are made to remove a posterior portion of corneal tissue 31 in preparation for the transplant. The resection incision 27 may be made using several different techniques. However, it should be noted that not all techniques provide the same quality of clinical results following the transplant. One technique is to make the resection incision 27 at a uniform distance from the posterior surface 33 of the cornea. U.S. patent application Ser. No. 11/375,542, the disclosure of which is incorporated herein by reference, discloses a method of making such an incision. By way of further example, techniques for making the resection incision 27 at a uniform distance from the anterior surface 25 are disclosed in U.S. Pat. No. 5,993,438, U.S. Pat. No. 6,730,074, and U.S. Patent Publication No. 20050245915. Other techniques known to skilled artisans may also be employed.

The thickness of the recipient cornea 21 is expressed as ‘a’. The depth, b, of the resection incision 27 shown in FIG. 1 is measured from the anterior surface 25 of the cornea. The ratio of the depth, b, to the thickness, a, is employed to determine the depth of the resection incision in the donor cornea as described below.

Similar to the resection incision 27, the sidecut 29 may also be formed through the different techniques that are known to skilled artisans.

The contact lens 23 is a rigid lens placed against the anterior surface 25, thereby forcing the anterior surface 25 to conform to the shape of the contact lens 23. Preferably, the contact lens 23 conforms the anterior surface 25 to a radial or planar shape. U.S. Pat. No. 5,549,632, which is incorporated herein by reference, describes making a laser incision by deforming the shape of the cornea. U.S. Pat. No. 6,863,667 and U.S. patent application Ser. No. 11/258,399, both of which are incorporated herein by reference, describe patient interface devices which may be used to align the surgical laser with the recipient cornea for purposes of making accurate incisions. Of course, the contact lens is not needed if the surgical laser system is capable of alignment with sufficient precision without use of the contact lens.

FIG. 2B illustrates the donor cornea 41 with a contact lens 43 placed against the anterior surface 45 of the donor cornea 41. The resection incision 47 and the sidecut 49 are made to remove the donor tissue 51 for grafting into the recipient cornea. The resection incision 47 and the sidecut 49 are preferably made using the same techniques employed to incise the recipient cornea. The ratio of the depth, d, of the resection incision 47 to the thickness, c, of the donor cornea 41 is preferably the same as the ratio that was set for the recipient cornea. By setting the depth of the resection incision 47 in this manner, compensation is made in the event that the donor cornea 41 swells due to fluid absorption following removal from the donor eye. Such swelling occurs because the donor cornea is resected from the donor eye in facility separate from the one in which the transplant is performed.

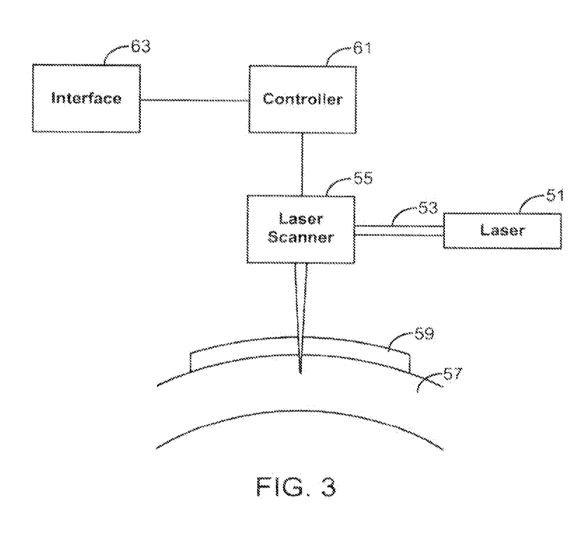

Referring to FIG. 3, a surgical system is shown which may be used to incise both a donor cornea or a recipient cornea. A femtosecond surgical laser 51 generates a pulsed laser beam 53 and directs that beam into the focusing assembly 55, which in turn focuses the pulsed beam 53 into the cornea 57. A contact lens 59 is placed over the cornea to deform the anterior corneal surface as described above. The controller 61 is a programmable computer which precisely controls the location of the beam focal point within the cornea 57 according to parameters received from the programmable surgeon interface 63. The interface 63 is programmed with a biomechanical model of the cornea and presents the surgeon with an incision depth range within which the surgeon may select the resection depth. The resection depth is received by the controller 61, which uses the focusing assembly 55 to make the resection incision at the resection depth.

A pulsed laser beam having ultra-short pulses, preferably in the femtosecond range, is employed to make the incisions. The laser may be of the type described in U.S. Pat. No. 4,764,930, producing an ultra-short pulsed beam as described in one or both of U.S. Pat. No. 5,984,916 and U.S. Pat. No. RE37,585. The disclosures of the aforementioned patents are incorporated herein by reference in their entirety. Commercial lasers capable of performing the incisions are available from IntraLase Corp. of Irvine, Calif.

Thus, a system and method of resecting corneal tissue are disclosed. While embodiments of this invention have been shown and described, it will be apparent to those skilled in the art that many more modifications are possible without departing from the inventive concepts herein. The invention, therefore, is not to be restricted except in the spirit of the following claims.

1-27. (canceled)

28. A method of resecting corneal tissue, the method comprising:
collecting physical data from a recipient cornea and from a donor cornea;
using a corneal biomechanical model informed by the physical data, determining the location of a minimum depth from an anterior surface of the recipient cornea sufficient to maintain post-operative stability of the recipient cornea when a posterior portion of corneal tissue is resected commencing at the minimum depth;
using the corneal biomechanical model informed by the physical data, determining the location of surface irregularities associated with appalanation of a first virtual contact lens onto the anterior surface of the recipient cornea, and determining a resection depth from an anterior surface of the recipient cornea which minimizes the surface irregularities and is at least as great as the minimum depth;
placing a contact lens having the same shape as the first virtual contact lens against the anterior surface of the recipient cornea, wherein the contact lens applanates the anterior surface, and conforms the anterior surface to the contact lens shape prior to making the resection incision using a surgical laser; and
making a resection incision using a pulsed surgical laser having ultra-short pulses to resect a posterior portion from the cornea, wherein the resection incision is made at the resection depth.

29. The method of claim 28, wherein the resection incision is at a uniform distance from one of a posterior surface or the anterior surface of the cornea.

30. The method of claim 28, further including comparing the minimum depth and the location of surface irregularities, and if they are substantially equivalent, redoing the step of determining the location of surface irregularities using information for a second virtual contact lens that has a larger radius of curvature than the first virtual contact lens and the step of placing comprises placing a contact lens having the same shape as the second virtual contact lens against the recipient anterior surface.

31. The method of claim 28, wherein the physical data includes thickness measurements made by optical coherence tomography.

32. The method of claim 28, wherein the minimum depth and location of surface irregularities are determined using the corneal biomechanical model also informed by a database of corneal measurements from multiple patients.

33. The method of claim 28, wherein the contact lens is part of a patient interface device.

34. The method of transplanting corneal tissue from a donor cornea to a recipient cornea, the method comprising:
collecting physical data from a recipient cornea and from a donor cornea;
using a corneal biomechanical model informed by the physical data and virtual stresses introduced into the cornea associated with appalanation of a first virtual contact lens onto the anterior surface of the recipient cornea, determining a recipient resection depth from an anterior surface of the recipient cornea;
determining a depth ratio of the recipient resection depth to a recipient thickness of ₜhe recipient cornea;
placing a contact lens having the same shape as the first virtual contact lens against the recipient anterior surface prior to making the recipient resection incision, wherein the contact lens applanates the recipient anterior surface and conforms it to a shape of the contact lens;
making a recipient resection incision for resecting a recipient posterior portion from the recipient cornea, wherein the recipient resection incision is made at the recipient resection depth using a pulsed surgical laser having ultra-short pulses;
making a donor resection incision for resecting a donor posterior portion from the donor cornea using the surgical laser;
making the donor resection incision at a donor resection depth from a donor anterior surface of the donor cornea, wherein the donor resection depth relative to a donor thickness of the donor cornea is equivalent to the depth ratio of the recipient resection depth to the recipient thickness; and
grafting the donor posterior portion into the recipient cornea.

35. The method of claim 34, wherein the recipient resection incision is at a uniform distance from one of a recipient posterior surface or the recipient anterior surface of the recipient cornea.

36. The method of claim 34, wherein the corneal biomechanical model determines the location of surface irregularities associated with appalanation of the first virtual contact lens onto the anterior surface of the recipient cornea and the recipient resection depth is selected to minimize the surface irregularities at the recipient resection incision.

37. The method of claim 34, wherein the corneal biomechanical model determines the location of a minimum depth from an anterior surface of the recipient cornea sufficient to maintain post-operative stability of the recipient cornea when a posterior portion of corneal tissue is resected commencing at the minimum depth.

38. The method of claim 37, wherein the corneal biomechanical model determines the location of surface irregularities associated with appalanation of the first virtual contact lens onto the anterior surface of the recipient cornea, the method further including comparing the minimum depth and the location of surface irregularities, and if they are substantially equivalent, redoing the step of determining the location of surface irregularities using information for a second virtual contact lens that has a larger radius of curvature than the first virtual contact lens and the step of placing comprises placing a contact lens having the same shape as the second virtual contact lens against the recipient anterior surface.

39. The method of claim 34, wherein the physical data includes thickness measurements made by optical coherence tomography.

40. The method of claim 34, wherein the minimum depth and location of surface irregularities are determined using the corneal biomechanical model also informed by a database of corneal measurements from multiple patients.

41. The method of claim 34, wherein the contact lens is part of a patient interface device.

42. A system of resecting corneal tissue, the system comprising:
an interface programmed with a biomechanical model of corneal tissue and physical data collected from a recipient cornea and from a donor cornea, wherein the interface provides a depth range using the biomechanical model for selection of a resection depth from an anterior corneal surface, the resection depth between a first depth based on the biomechanical model and a second depth based on the biomechanical model, the first depth corresponding to maintaining a post-operative stability of a recipient cornea when a posterior portion of corneal tissue is resected commencing at the minimum depth, the second depth corresponding to minimizing surface irregularities at the resection depth associated with appalanation of a first virtual contact lens onto the anterior surface of the recipient cornea;
a contact lens, wherein the contact lens is placed against the anterior surface and applanates the anterior surface and conforms it to the contact lens shape;
a surgical laser, wherein the surgical laser emits a pulsed laser beam;
a focusing assembly, wherein the focusing assembly focuses the pulsed laser beam into the recipient cornea; and
a controller, wherein the controller receives the resection depth from the interface, moves a focal point of the pulsed laser beam within the recipient cornea using the focusing assembly, and directs the focal point of the pulsed laser beam to make a resection incision for resecting a portion of the cornea, wherein the resection incision is made at the resection depth.

43. The system of claim 42, wherein the resection incision is at a uniform distance from one of a posterior surface of the recipient cornea or the anterior surface.

44. The system of claim 42, wherein the controller controls the surgical laser to make the resection incision for resecting a posterior portion of the recipient cornea.
